(19) United States
(12) Patent Application Publication
(10) Pub. No.: US 2010/0203595 A1
(43) Pub. Date: Aug. 12, 2010

(54) THERMAL CYCLING APPARATUS AND PROCESS

(76) Inventors: David Ward, Teeside (GB); Nelson Nazareth, Cambridgeshire (GB)

Correspondence Address: J. BRUCE HOOFNAGLE, P. O. BOX 370, LISBON, MD, 21765-0370, US

(21) Appl. No.: 12/450,028

(22) PCT Filed: Mar. 6, 2008
(86) PCT No.: PCT/GB2008/000775

§ 371 Date: Apr. 20, 2010

(30) Foreign Application Priority Data
Mar. 8, 2007 (GB) 0704490.2

(51) Int. Cl. C12P 19/34; F28F 13/06; F25B 21/04; C12M 1/00; C12M 1/34

(52) U.S. Cl. 435/91.2; 165/135; 62/3.3; 165/181; 435/303.1; 435/287.2

(57) ABSTRACT

A thermal cycling apparatus 9 and process includes at least one reaction vessel 14 which is associated with a thermoelectric cooler 12 (TEC), such as a Peltier cell, and arranged to provide both heating and cooling of the reaction vessel. A first side of the TEC 12 is associated with the at least one reaction vessel 14 and a second side of the TEC is arranged in use to be maintained at a temperature intermediate the highest temperature and the lowest temperature used in a thermal cycling operation. Electric current is supplied to the TEC 12 in one direction whereby the said first side becomes hotter than the second side, and then in the other direction whereby the first side becomes cooler than the second side.

This application claims the benefit of International Application No. PCT/GB2008/000775, filed on Mar. 6, 2008, which, in turn, claims the benefit of UK Application No. GB 0704490.2, filed on Mar. 8, 2007, both of which are incorporated herein by reference thereto.

### FIELD OF THE INVENTION

This invention relates to thermal cycling apparatus and processes, such as biological, chemical and biochemical processes and apparatus. It is particularly concerned with processes and apparatus in which controlled heating, and possibly cooling, has to be applied to a substance, such as a sample. A typical biological process is the Polymerase Chain Reaction, hereinafter referred to as “PCR.” PCR processes are described in U.S. Pat. Nos. 4,683,195 and 4,683,202, both of which issued on Jul. 28, 1987. However the present invention is by no means limited in application to PCR processes.

### BACKGROUND OF THE INVENTION

This application includes matter which is additional to matter disclosed in the above-noted UK Application No. GB 0704692.3.

In the case of certain biological, chemical and biochemical processes, hereinafter referred to as BCBC, such as, for example, PCR processes, the accurate measurement and control of process temperatures is critical in maintaining the specificity and efficiency of the process. In apparatus for performing such processes, the speed, specificity, sensitivity and reproducibility of reactions performed is readily reduced by limitations in temperature control performance and by restrictions to the transfer of heat energy into and out of the reaction vessel. Therefore, there is need for providing improved temperature control and hence improved performance in such processes and apparatus.

In this application, the word “vessel” refers to any device capable of holding a substance, or a sample thereof, to be processed, and may accordingly include a well, a tube (open or closed), and a slide, perhaps in the form of a silicon chip or a tray. The invention is particularly concerned with microtiter vessels in well form.

In this application, the term “thermal cycling” is used to refer to the control of a reaction vessel whereby the vessel is heated, and cycled through, a number of temperatures for a specified period of time. In most cases it is desirable for the process to be completed in as short a time as possible. This is particularly the case where PCR is being employed in the identification of a pathogen, when three temperatures are employed, namely—the upper denaturing temperature, the lower, recombination temperature, and the extension temperature, which is intermediate the upper and lower temperatures. Thus, there is need for a thermal cycling process where the required temperatures are reached and maintained as accurately and rapidly as possible so that the times between successive temperatures are as low as possible.

Thermal Cycling speed is limited by a number of closely interrelated factors as follows:

Thermal conductivity of the reaction vessel. The lower the thermal conductivity of the reaction vessel the longer it will take to transfer heat to and from the contents of the vessel.

Likewise, the thermal conductivity of any interface between the heat source and the heat sink on the one hand and the vessel on the other.

The larger the specific heat capacity of the vessel the more thermal energy must be transferred to and from the vessel in order for a given temperature change to occur.

The greater the delta temperature (the difference in temperature between source or sink on the one hand and the vessel on the other) the faster the heat transfer to and from the vessel content can take place. This may be assisted by using a high wattage heater and increasing the capacity to remove heat thus enabling the highest delta temperature possible to be maintained.

In the past, the approach to thermal cycling in the BCBC context has been to rely upon a discrete heating element and a discrete cooling element to heat and cool the reaction vessels. More or less implicit in this is that rapid heat transfer in and out requires a powerful heater and a massive heat sink.

Also, an attempt has been made to improve the thermal mass of the system by reducing the specific heat capacity and increasing the thermal conductivity of the reaction plate by lining it with silver or boron nitride. However this has a small impact on the overall thermal mass of the system as a whole and as such it is an expensive modification for little benefit.

However, most of the thermal lag of the instrument is actually in the heater and the cooler elements themselves.

Therefore, there is a need for a thermal cycling apparatus and processes with rapid heat transfer, which uses a comparatively moderate sized heater and heat sink to facilitate performance of thermal cycling with minimal or no thermal lag.

### SUMMARY OF THE INVENTION

It is, therefore, an object of this invention to provide for improved temperature control and hence improved performance in BCBC processes and apparatus.

Another object of this invention is to provide a thermal cycling process where the required temperatures are reached and maintained as accurately and rapidly as possible so that the times between the successive temperatures are as low as possible.

Still another object of this invention is to provide a thermal cycling apparatus and processes with rapid heat transfer, which uses a comparatively moderate sized heater and heat sink to facilitate performance of a thermal cycling process with minimal or no thermal lag.

With these and other objects in mind, this invention contemplates a thermal cycling process and apparatus carried out in at least one reaction vessel and employs a thermoelectric cooler (TEC) to provide both heating and cooling of each of the at least one reaction vessels. As is well known, in a TEC, an electric supply to a differing material junction, or more normally a plurality thereof, causes a thermal disparity to arise between a so called hot side and a cold side. An example of a typical TEC is a Peltier effect cell.

According to an important feature of the invention, there may be provided one TEC per reaction vessel and the reaction vessel may be a microtiter vessel, i.e., one reaction vessel in an array of such vessels, typically a 12×8 array or an integer multiple thereof.

TEC devices operate at their highest efficiency when both sides, i.e. hot and cold sides, of the TEC are at the same temperature. As the hot side of the TEC increases in temperature and the cold side of the TEC decreases in temperature, the heating and cooling efficiency decreases. This is illustrated in GRAPH A below.

According to an important feature of the invention, the thermal cycling process and apparatus may be arranged such that, in operation, one side of the TEC is always kept at an intermediate temperature, which is also intermediate the upper temperature and the lower temperature used in the thermal cycling operation.

Ideally, in the PCR context, the intermediate temperature chosen is slightly below the extension temperature. The extension temperature is the temperature at which the enzyme employed in the PCR process operates upon the DNA free strand and is generally constant for a specific enzyme. PCR is at its most efficient when the cycle dwells at the extension temperature for the known period of time within which the “extension” occurs. Typically for PCR the intermediate temperature is 72-74° C.

In normal operating conditions, the extension temperature is above ambient. This confers a considerable advantage in the present instance. When one side of the TEC is held at a temperature above ambient, such as the extension temperature, the TEC can be so operated as to “pivot” around that temperature. This inevitably increases thermal cycling characteristics since the highest efficiency of the device is achieved when cooling and heating from this holding or intermediate temperature.

According to another important feature of the invention, the side of the TEC to be maintained at the intermediate temperature may be arranged to be in contact with, or even preferably attached to, a heat exchange block. Preferably the heat exchange block is the heat removal module (HRM) described in UK Patent Application No. 0626065.7, filed on Sep. 19, 2006, and UK Patent Application No. 0718250, filed on May 31, 2007, and includes a block of thermally conductive material having therein a channel array adapted for the flow of a heat transfer liquid.

The channel array may be in labyrinthine, serpentine form. The block may be formed of two mating plates with the labyrinth formed in one or both mating surfaces, perhaps by routing or milling, with a suitable sealant employed between the plates. Alternatively the module is a single block and the labyrinth formed by drilling therethrough and then blocking unwanted exits and routes with stoppers such as grub screws. In another alternative, the block may be molded, for example of a powdered metal or carbon or carbon or boron loaded plastics material around a former for the serpentine channel. The serpentine channel may in this case be a preformed metal, e.g., a copper tube with a 2-3 mm bore. Alternatively, the channel array may include a suite of parallel channels with inlet and outlet manifolds. In this instance, either the construction of the manifolds, or the power of the coolant pump, may be arranged to ensure that coolant flows in each channel.

Additionally, or alternatively, the block may include a heat pipe, that is a sealed metal tube containing wicking and a small quantity of a liquid such as water.

The material that the block is formed of can depend upon the context and ease of use and economic considerations, with copper, aluminum alloy, silver, or gold, boron nitride, diamond and graphite among the possibilities.

The liquid may be water, preferably deionized water with an antioxidant addition. A typical example of such a coolant liquid is FluidXP+, which is available from Integrity PC Systems & Technologies, Inc of Riverdale, Calif. USA.

The heat exchange block may, however, include any device capable of being maintained at a constant temperature and to which the TEC can be mounted, for example, by soldering. A metal heat store would thus provide another example.

The arrangement, in the PCR context, is: (1) the heat exchange block is maintained at a constant temperature, using the liquid flowing therein, the temperature being at or slightly below the PCR extension temperature and in the normal operating context somewhat above ambient; (2) a first face of the TEC being in contact with the heat exchange block, with the temperature of that face being held substantially constant; (3) an electric current supplied to the TEC in one direction causes a second face of the TEC to heat up with respect to the first face; and (4) reversal of the electric current supplied to the TEC causes the second face of the TEC to cool with respect to the first face.

Importantly, in a 12×8 array, this arrangement facilitates individual control of each vessel.

The second face of the TEC may be arranged to be in contact with a holding cup arranged to accept snugly a reaction vessel and to transfer heat thereto and therefrom. Preferably the holding cup is attached to the second face. The holding cup may be formed, perhaps punched, from sheet metal or fabricated from metal, metalloid, or thermally conductive glass or plastics material. Typical metals include silver, gold, aluminum and tin. They may be anodized where deemed necessary. Ideally, the holder is formed so as to have a thermal conductivity greater than 1.5 W/mK.

The use of a temperature measurement device, such as, for example, a thermistor, may be avoided by prior or periodic calibration of the apparatus. Where, however, this remains desirable, a temperature measurement device may be incorporated in the holding cup, or in or above the lid, where a lid is employed. The temperature measurement device may be included, of course, in the TEC electrical supply circuit to provide means for temperature control. The array of vessels may be monitored sequentially using a high speed multiplexer or, concurrently, using an array of temperature controllers. Where contact thermometry is not desired or preferred, non-contact thermometry may be employed using a thermal camera or pyrometer device, again, either sequentially or continuously.

Control gear may, if required, be incorporated to provide the required functionality. The control gear allows the operating current to be applied to a varying degree (preferentially by pulse width modulation) with the additional capability of reversing the polarity of the supplied voltage to facilitate the heating or cooling of the TEC. Insofar as this requires a high current supply, the TECs may be divided into manageable groups, with each group then being connected individually to the main power supply.

Temperature measurement devices are advantageously incorporated. Ideally these include a sensor, such as a thermistor, to the TEC, or in/on the cup, whereby the time for each sample to reach the required temperatures can be monitored and the current polarity switched after any required dwell, to minimize reaction time.

The electrical circuitry may also incorporate means enabling the detection and shutting down of any reaction vessel deemed to be failing. Too high a speed of temperature transition can mean absence of a vessel while too low a speed implies an error with the control gear or the TEC.

The preferred vessel construction for this context is a well in which there is a high surface-to-volume ratio associated with the vessel reaction chamber, and the vessel wall is highly thermally conductive. A vessel having a reaction chamber, which includes a tube of capillary, or slightly greater than capillary, dimensions to an aqueous solution content, and an aspect ratio of between three and ten to one, is preferred. The vessel may be formed of a polymer, preferably one which is non-biologically reactive, loaded with a thermally conductive material such as carbon or boron nitride. Advantageously, the vessel has the thinnest wall thickness possible consistent with structural and handling integrity in the circumstances of use. For example, a microtiter vessel wall formed, as described above, may have a wall thickness between about 0.2 and 1.0 mm.

This arrangement has an important advantage over arrangements employing electrically conductive polymers in the construction of the vessels, such as those described in UK Patent No. 2333250, which was published on Jun. 5, 2002, namely that the danger is avoided of an electrical field interfering with the reaction occurring in the reaction chamber. This deleterious effect has been noted particularly in the case of PCR, though it may well apply to other ionic reactions.

However, it is particularly useful, if not important, for such vessels to be provided with lids, which fit relatively tightly thereto. Lids serve the purpose of preventing content contamination or loss, and of retaining the heating and cooling to within the vessel reaction chamber. Such lids are generally provided with a translucent portion adjacent the reaction chamber, whereby the progress of a reaction can be monitored optically. It is also, accordingly, valuable for the translucent portion to be maintained free of condensation. The lid is preferably arranged so that when a standard reaction sample volume is placed in the vessel the free space between the lid and the sample is minimal.

Maintaining the translucent portion of the lid free of condensation, and minimizing heat loss through the lid, can be improved where necessary by heating the lid independently of the vessel. An electrical coil may be incorporated for this purpose or, indeed, the lid may be, in part, constructed of an electrically conductive polymer (ecp) and arranged to receive the necessary heating current.

The lid may be arranged in use to follow a thermal profile of the reaction contents, but at an offset temperature. Thus, for a reaction chamber temperature cycle of 56-72-95° C., the lid cycle might be of the order of 56-72-105° C.

Optical monitoring may be effected employing the apparatus and method described in UK Patent No. 2424381, which was published on Jun. 27, 2007. This describes a method and apparatus for real time monitoring optically of chemical or biological reactions in a plurality of reaction vessels in an array of receiving stations, wherein a beam of laser light is directed via a mirror array into one or more of the vessels to excite the contents thereof; and any resultant light emitted from the reactants in the vessels is directed via mirrors and a diffraction grating to a multi-anode photomultiplier tube (MAPMT).

An alternative optical monitor system includes a printed circuit board (PCB) arranged for presentation above the reaction vessels, the PCB holding an array of light emitting diodes (LEDs) selected so as to be within the excitation spectrum of the vessel contents under interrogation and arranged for the direction of light into the vessel. The PCB also has a foramen arranged to permit the passage of vessel content light emission spectra, and the system also including detector apparatus arranged to detect the emission spectra, and filter means to block the path of excitation spectra to the detector.

Preferably the LEDs are arranged to emit light at the blue end of the optical spectrum, typically at a wavelength of 470 nm or above. One suitable detector apparatus may comprise a fresnel lens arranged to direct the light onto an XY scanning mirror set and thereby into a detector such as a PMT, APD (avalanche photo-diode), CCD (charge couple device), LDR (light dependent resistor) or a photovoltaic cell. The PMT may be single cell or, if the emission beam is split into a spectrum, an array thereof. The filter means may comprise an optical filter placed, for example, across the foramen or software associated with the detector. Where, as will usually be the case, there is a lid to the vessel, the optical monitor system is arranged for light path association therewith.

Typically, thermal cycling reaction apparatus is arranged to receive, in stations, a standard array of 96, or an integer multiple thereof, microtiter reaction vessels in a rectangular array, usually having 12×8 such stations. This is a preferred arrangement for the present invention. In other words, it has been discovered that it is possible to construct an array of Peltier cells attached to a heat transfer block and each having a 9.0 mm square, or even smaller, footprint.

Also, it has been discovered that, with a heat removal module as described above, a mean vessel cooling rate of 18° C. per second can be achieved, peaking at 24° C. per second.

In another embodiment, the heat exchange block may be constructed to be directly heated using a heater mat or by having the block itself become part of the heater, for example, by using an electrically conducting polymer. As an example, a graphite/boron nitride loaded block of plastic can be molded with an electrical resistance (determined by the graphite loading) such that the block can be connected to a power supply and used to perform useful resistive heating.

### BRIEF DESCRIPTION OF THE DRAWINGS

Embodiments of the present invention will now be described by way of example with reference to the accompanying drawings, of which:

FIG. 1 illustrates a thermal cycling apparatus having a thermoelectric cooler (TEC), such as, for example, a Peltier cell, mounted upon a heat exchange block and carrying a vessel holder and vessel in accordance with certain principles of the invention;

FIG. 2 is a schematic sectional diagram of an array of TECs on the heat exchange block of FIG. 1 in accordance with certain principles of the invention;

FIGS. 3 and 4 illustrate alternative constructions of a heat transfer block, in accordance with certain principles of the invention;

FIGS. 5 and 6 illustrate alternative optical monitoring systems for monitoring the condition of a sample substance during a thermal cycling process, in accordance with certain principles of the invention;

FIGS. 7a, 7b and 7c illustrate a suite of series-, parallel-, and individually-connected TECs, respectively, arranged for use during a thermal cycling process, in accordance with certain principles of the invention:

FIG. 8 illustrates a cooling arrangement for the heat exchange block of FIG. 1, in accordance with certain principles of the invention;

FIG. 9 illustrates an electrical control circuit and a heat sink associated with the operation of thermal cycling apparatus of FIG. 1, in accordance with certain principles of the invention; and

FIGS. 10a, 10b and 10c illustrate a series of waveforms associated with the optical monitoring systems of FIGS. 5 and 6 during the filtering out of an excitation spectrum from data-bearing spectra to be provided to a detector, in accordance with certain principles of the invention.

### DESCRIPTION OF THE PREFERRED EMBODIMENTS

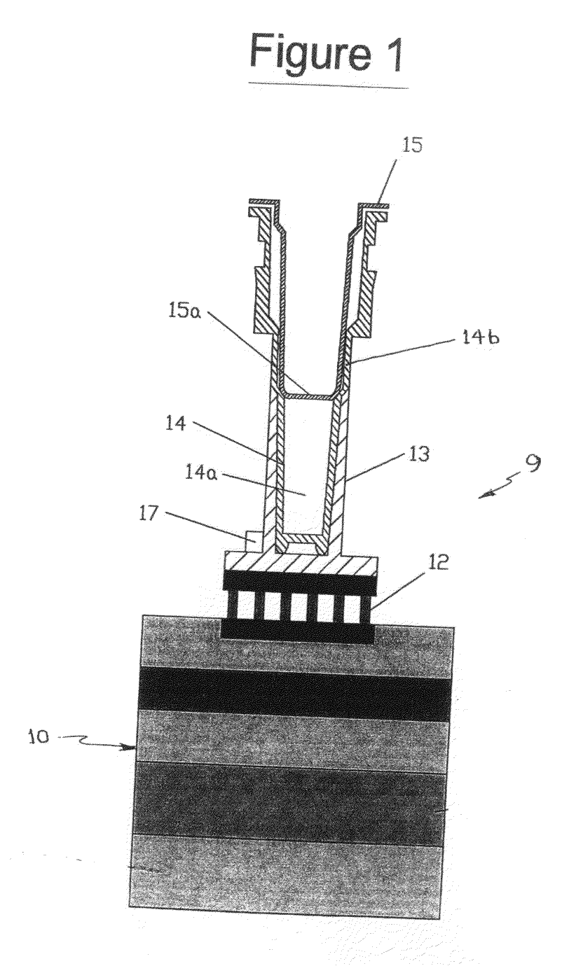

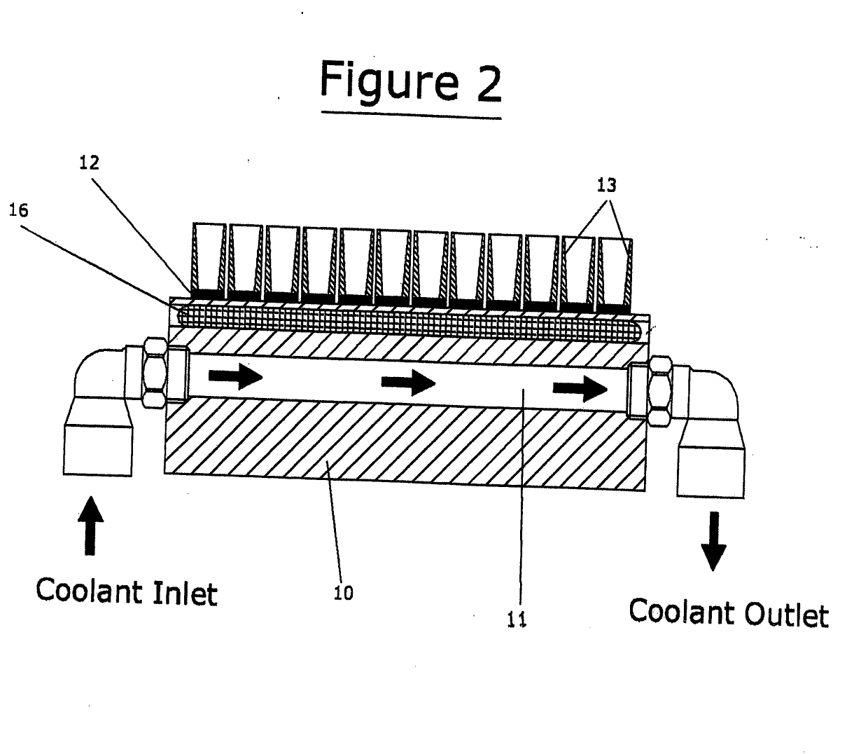

As shown in FIGS. 1 and 2, a thermal cycling apparatus 9 includes a thermally conductive heat removal module (HRM) 10, also referred to as a heat exchange block, and a duct, having coolant channels 11 (FIG. 2), for conveying a coolant, or heat transfer, liquid. An array of thermoelectric coolers 12 (TECs), such as, for example, Peltier cells, are attached at a first face thereof to the HRM 10 in such a manner that there is a good thermal conductive relationship therebetween. A thermally conductive receiving cup 13 is mounted to a second face of each TEC 12. The cup 13 is arranged to act as a receiving station for a reaction vessel 14, and is accordingly constructed to envelop the vessel in contiguous relationship therewith.

Each of the TECs 12 and the cups 13 incorporate temperature sensors (not shown). The temperature sensors are associated, in a control circuit, with a high speed multiplexer (not shown) enabling rapid reading of the reaction status in each vessel 14, and arranged to measure the time taken for each vessel to reach both the upper and lower temperatures in, for example, a PCR cycle.

The HRM 10 and the cups 13 are formed of a low specific heat capacity, highly thermally conductive material with a high resistance to oxidation. A typical example of such material, and having also the advantage of relatively low cost, is anodized aluminum alloy.

The HRM 10 extends somewhat beyond the footprint of the vessel array to allow a near identical heat removal capability to each TEC 12, an example of which, as noted above, is a Peltier cell.

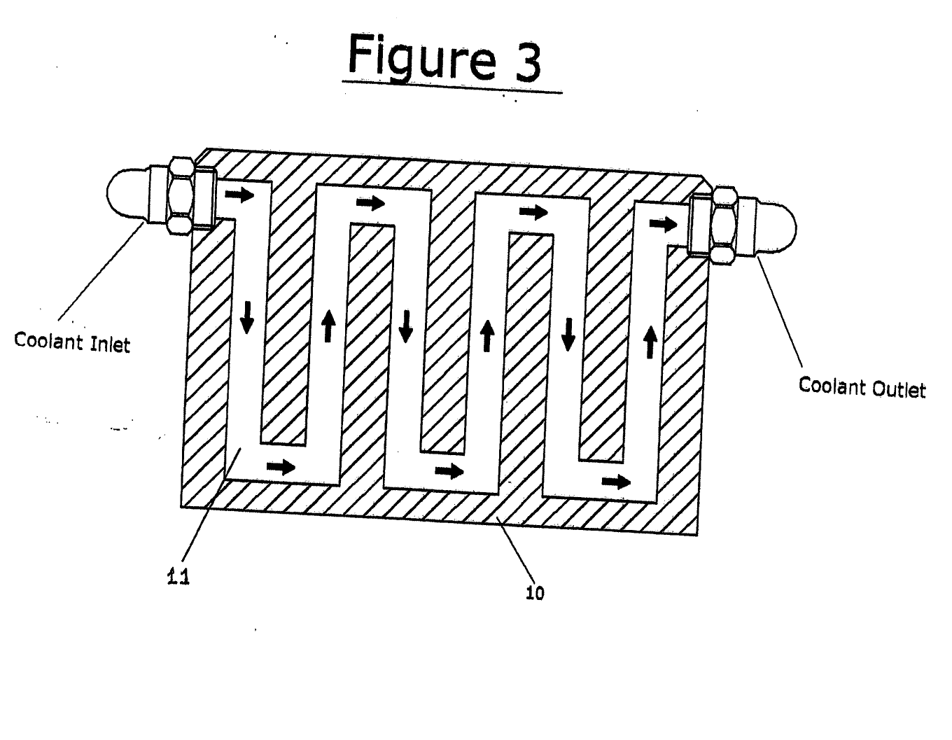

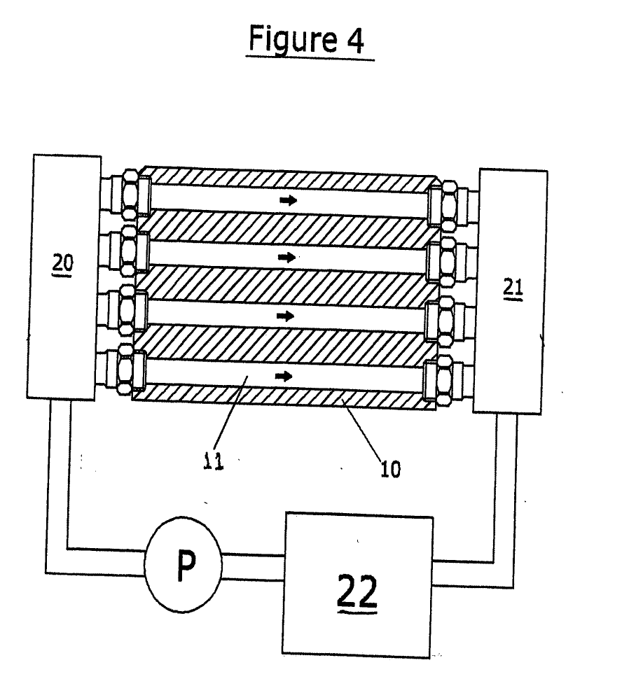

The duct, having the coolant channels 11, is associated with a heat exchanger, for example, a heat exchanger 22 (FIG. 4), and a pump, for example, a pump P (FIG. 4), whereby the temperature of the coolant liquid, caused to flow therein, is controlled.

The vessel 14 has a reaction chamber 14a and a lid reception portion 14b, in which fits a lid 15 having a transparent lower face 15a permitting optical monitoring of the reaction in the reaction chamber 14a. The reaction chamber 14a has a high surface-to-volume ratio, with a bore slightly greater than capillary for an aqueous solution and an aspect ratio of eight. The vessel 14 is formed of a carbon loaded polymer and has a wall thickness of 0.4 mm whereby it is inexpensive and highly suitable as a consumable.

The lid 15 fits into the lid reception portion 14b of the vessel 14 in such a way as to minimize the air gap between the window 15a and the upper level of a standard sample located in the reaction chamber 14a. The cup 13 extends upward to the base of the reception portion 14b of the vessel 14, thereby establishing a level to which a standard sample should fill the reaction chamber 14a, with an air gap between the sample and the lid being minimal.

A thermistor 17, which is a temperature measuring device, is mounted on the cup 13 to measure the temperature thereof.

A particularly suitable reaction vessel 14 includes a working or reaction portion 8 mm long with a mean bore of 2.5 mm, a contact portion of approximately 4.0 mm outside diameter and 3.0 mm length and a funnel portion of 6.0 mm mean outside diameter and 7.0 mm length. The vessel 14 is formed of a thermally conductive material. The thermally conductive material may comprise a carbon based filler such as Buckminster fullerine tubes or balls, carbon flake or powder within a polypropylene matrix. Typically, the carbon content is up to 70% by weight, with 10% being carbon black and the remainder being graphite. The total wall thickness of the vessel 14 is of the order of 0.3 mm. To avoid spillage and filling problems both parts of the vessel 14 have a taper of 1.5° from vessel axis down towards the base thereof.

The TECs 12 are arranged to have a footprint just less than 9.0 mm×9.0 mm thus allowing their use in a 96 vessel (12×8) microtiter vessel array, and permitting a single reaction vessel 14, or group of reaction vessels, to be thermally cycled separately from other reaction vessels or groups of reaction vessels.

As shown in FIG. 2, the HRM 10 includes a heat pipe 16. This optional item assists in ensuring homogeneity of the temperature of the HRM 10 throughout the block. As the TEC 12 performs resistive heating, as well as pumping heat between the two faces thereof, excess resistive heat is generated, which is dissipated by the HRM 10 and an associated heat sink 128 (FIG. 9). In cycling an array of vessels 14 independently, instances are likely to arise where one TEC 14 is in the heating phase of a cycle while an adjacent TEC is in the cooling phase. The heat pipe 16, by transferring heat anywhere within the HRM 10, minimizes heat exchange between the two TECs.

The construction of the HRM 10 is shown more clearly in FIG. 2, which is a diagrammatic cross section of a side elevation thereof. The coolant channels 11 and the heat pipes 16 are in parallel array and, in contradistinction to the illustration in FIG. 2, extend below each row of eight TECs 12. The channels 11 and the heat pipes 16 may be arrayed transverse one to another or, as illustrated, extend below each row of twelve TECs 12, but it is believed that the parallel array described above is optimum. In this microtiter vessel context, a bore, or channel 19 (FIG. 2), formed in the HRM 10 for receipt of the heat pipe 16, like that of the channels 11, is 3.0 mm.

FIGS. 3 and 4 illustrate alternative channel arrangements within the HRM 10. In FIG. 3, there is a single channel 11 following a serpentine path. In FIG. 4, there is an array of parallel channels 11 connected between an inlet manifold 20 and an outlet manifold 21. Also shown is the heat exchanger 22 and the pump P, which completes the coolant circuit. This arrangement is also applicable to the arrangements of FIGS. 1, 2 and 3. The advantage of using the serpentine channel array of FIG. 3 over the parallel array of FIG. 4 may be the assurance of a constant flow throughout. A disadvantage, which may be overcome by the heat pipes 16, is a variation of temperature over the length of the channel 11.

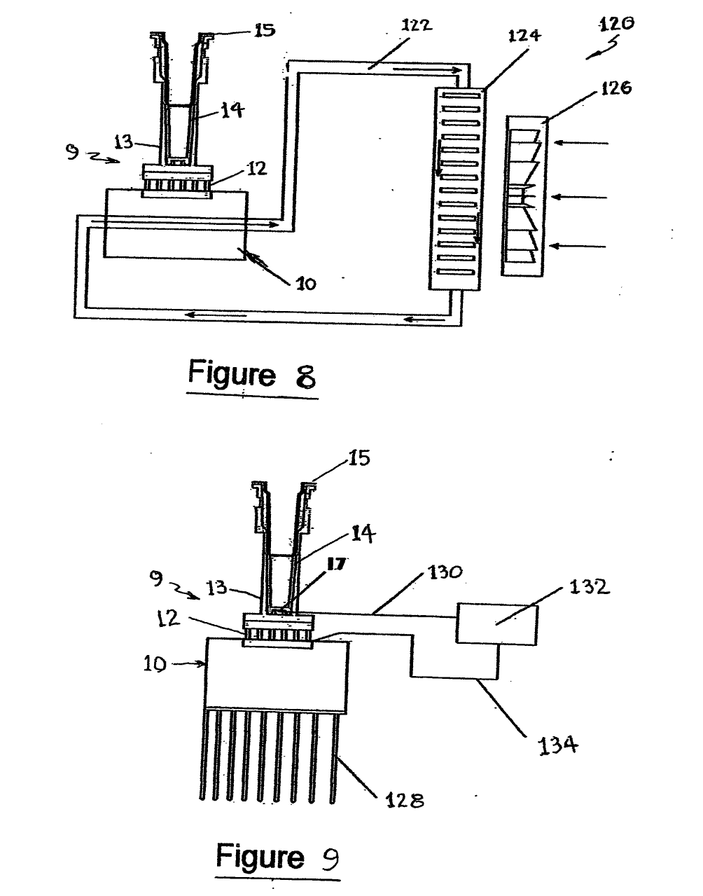

As shown in FIG. 8, an alternative cooling system 120 includes a conduit 122 for circulating the coolant liquid through a radiator 124, and which passes through the HRM 10. A fan 126 is located adjacent the radiator 124 to draw cooling air through the radiator.

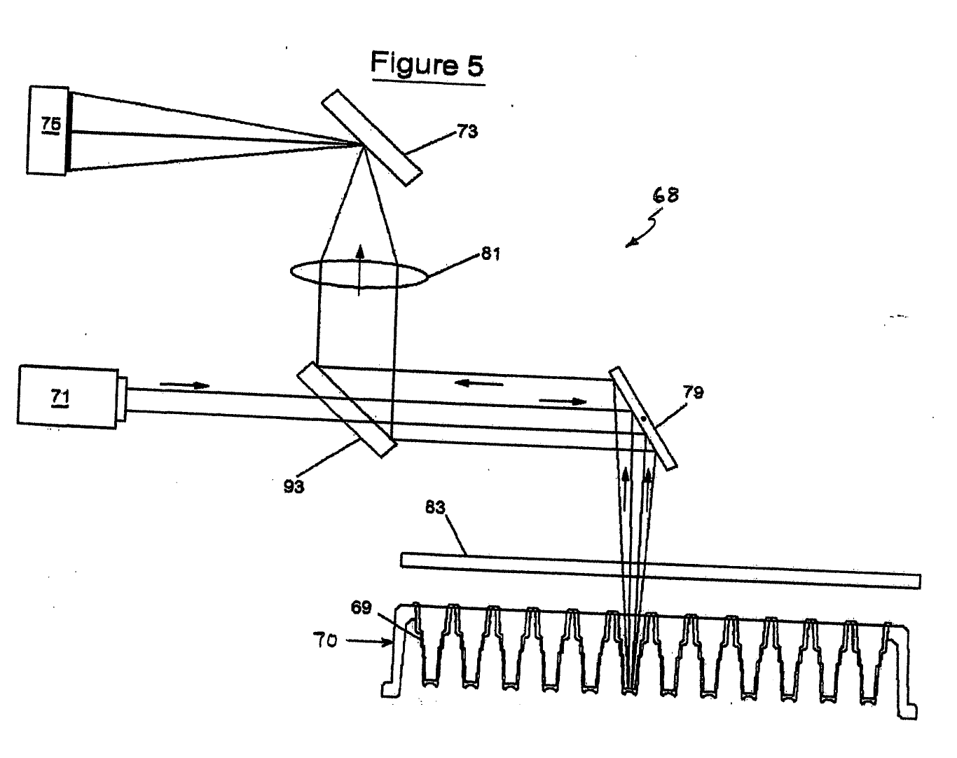

An optical monitoring system 68 for a reaction apparatus 70 is illustrated in FIG. 5. The reaction apparatus 70 includes a plurality of receiving stations, with each station receiving a reaction vessel 69 in which a reaction may take place. The system 68 includes at least one laser light source 71, a scanning apparatus 79 for directing light to the reaction vessels 69 in the receiving stations and for receiving radiation emitted from the reaction vessels and directing the radiation via a diffraction grating 73 to a multi-anode photomultiplier tube assembly 75 operating in a photon counting mode. A foraminous mirror 93 contains a foramen at 45 degrees to the plane of the mirror, permitting laser light to pass through it to the vessels 69. The majority of diverging emitted light from the vessels 69 is reflected to the diffraction grating 73, since at this point the emitted light beam is of much greater diameter than the foramen.

The multi-anode photomultiplier tube assembly 75 includes a multi-anode photomultiplier tube (MAPMT) with a 32 pixel array over which radiation from around 510 to 720 nm is dispersed. Radiation emitted by the reaction vessel contents is dispersed over the pixels of the MAPMT by the diffraction grating 73 such that the wavelength range of the radiation impinging on a photocathode of the MAPMT correlates with the position of the photocathode in the MAPMT.

The light source 71 is a diode pumped solid state laser (DPSS Laser) which is smaller and lighter than conventional gas lasers typically used in optical monitoring systems.

The scanning apparatus 79 includes one or more planar rotatable mirrors, for clarity only one such mirror is illustrated. These are motor driven and controlled by means which are omitted from the drawings for clarity. The system of mirrors can be configured to direct the light from the laser to any receiving station. Radiation emitted is returned to the foraminous mirror 93 which reflects the majority of the emitted radiation through a lens 81 which focuses the radiation upon the diffraction grating 73.

A Fresnel lens 83 is interposed between the rotatable mirrors, e.g. mirror 79, and the receiving stations to ensure verticality of the light entering each reaction vessel 69.

Referring to FIG. 1, in use of the thermal cycling apparatus 9, with a sample to be subjected to polymerase chain reaction amplification, coolant is passed through the coolant channels 11 of the duct of the HRM 10 to maintain the lower face of the TEC 12 at a temperature slightly lower than the PCR extension temperature (typically 72-74° C.). This allows the TEC 12 to “thermally pivot” around this set point temperature. Then the polarity of the current supplied to the TEC 12 is switched alternately at the rate required to effect PCR until the optical array detects the change in returned optical wavelength, which will signify that sufficient amplification has been achieved. The effect of this pivoting action is illustrated in TABLE A and GRAPH B below.

TABLE A

| Time | Hot no | Cold | | | | |
|---|---|---|---|---|---|---|
| base (s) | HRM | No HRM | dT | Hot HRM | Cold HRM | dT Heat |
| 0 | 25 | 25 | 0 | 70 | 70 | 0 |
| 0.57 | 39.7 | 2.6 | 14.7 | 107.1 | 70 | 37.1 |
| 1.4 | 51 | −7 | 11.3 | 128 | 70 | 20.9 |
| 3 | 55.1 | −11.8 | 4.1 | 136.9 | 70 | 8.9 |
| 4 | 55.4 | −13.2 | 0.3 | 138.6 | 70 | 1.7 |
| 5 | 55.7 | −14.2 | 0.3 | 139.9 | 70 | 1.3 |
| 6 | 56 | −14.5 | 0.3 | 140.5 | 70 | 0.6 |
| 7 | 55.7 | −14.5 | −0.3 | 140.2 | 70 | −0.3 |
| 8 | 55.7 | −14.5 | 0 | 140.2 | 70 | 0 |
| 9 | 55.7 | −14.5 | 0 | 140.2 | 70 | 0 |
| 10 | 55.1 | −14.3 | −0.6 | 139.4 | 70 | −0.8 |

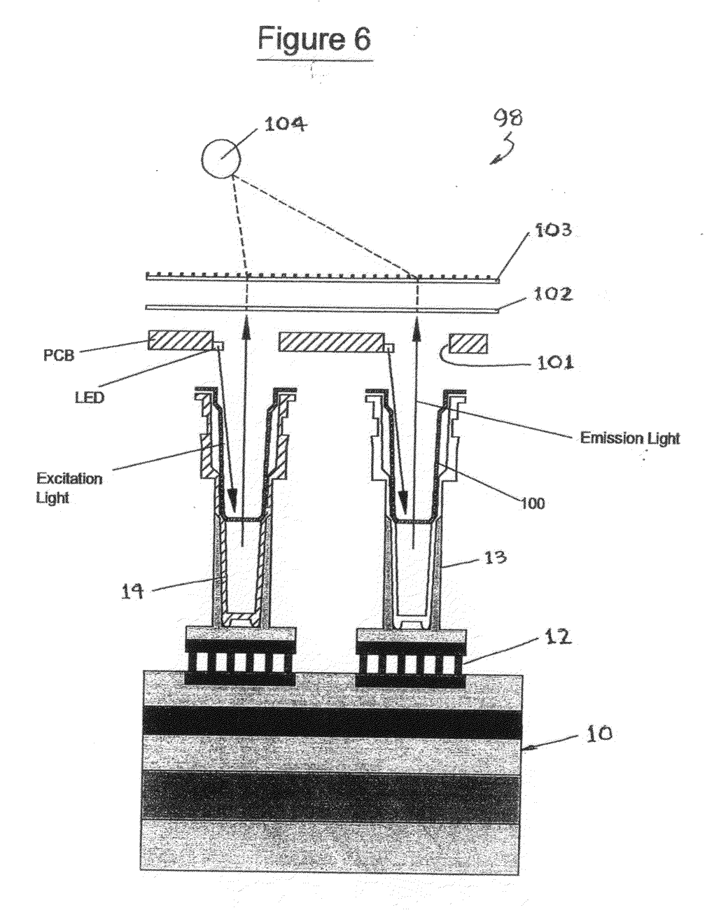

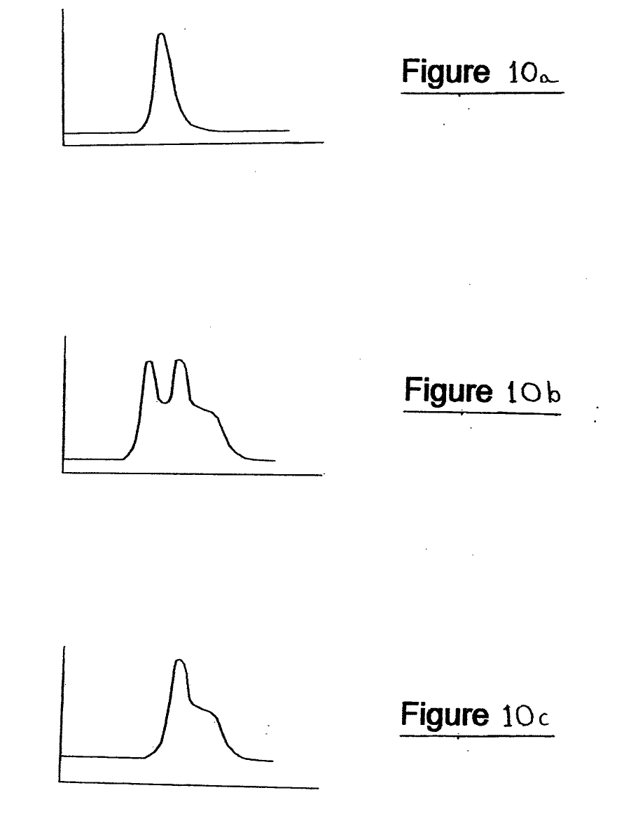

The thermal cycling apparatus 9 (FIG. 1) also includes software or firmware capable of characterizing the heating and cooling speeds of the TECs 12 to allow the control gear to modify its control loop and permit all TECs to operate as if identical. In the operation described above, discrete filters are placed into the optical path, between the sample of the substance within the reaction chamber 14a and, for example, the detector 104 (FIG. 6). In instances where a number of excitation sources, one example of which are the LEDs (FIG. 6), are used, a set of filters would have to be used and cycled to filter the different excitation spectra. Instead of requiring the set of filters noted above, and in accordance with certain principles of the invention, software facilitates the filtering of the excitation spectra from the return signal, leaving only the data-bearing spectrum to be fed to the detector 104. This principle is illustrated in FIGS. 10a, 10b and 10c, wherein the waveform of FIG. 10a represents the excitation spectra, the waveform of FIG. 10b represents the data-bearing spectrum and the excitation spectra, and the waveform of FIG. 10c represents the data-bearing spectrum, with the excitation spectra having been filtered out.

The thermal cycling apparatus 9 also includes means for enabling the detection and shut down of any individually failed reaction vessels 14 by monitoring the speed of temperature transition (too high speed means no reaction vessel present). Where the reaction speed is not as fast as expected the reaction vessel position may be disabled or flagged as an error. The means for enabling is incorporated in the power circuit of the TEC control circuit, whereby AC current (less than 100 mA) is supplied to the TEC and the resistance of the TEC is measured. An increase in the AC resistance of the TEC is interpreted as the degradation of the TEC and will gradually increase throughout the life of the TEC. When a resistance threshold is exceeded, the TEC has failed and will no longer be used.

As shown, the thermistor 17 is coupled through a temperature response path 130 to an electrical control circuit 132, which serves as a temperature controller. In response to the output of the thermistor 17, the electrical control circuit 132 supplies current, established at a level responsive to the output of the thermistor, through a current feed path 134 to the TEC, thereby providing an arrangement for the control of the electric current to the TEC.

An alternative embodiment of an optical monitoring system 98 is illustrated in FIG. 6. In this embodiment, a printed circuit board (PCB) is presented to the reaction vessel lids 100, the PCB holding an array of light emitting diodes (LED) selected to emit light at 470 nm and arranged for the light thereof to be directed through the translucent portion of the lid 100. A foramen 101 in the PCB is fitted with an optical filter 102, to filter the excitation spectrum, whereby only the emission spectra, and not the excitation spectra, is allowed to pass. A Fresnel lens 103 alters the path of the emission light emerging from the plurality of vessels 14, and onto a detector 104 in the form of a photomultiplier tube (PMT).

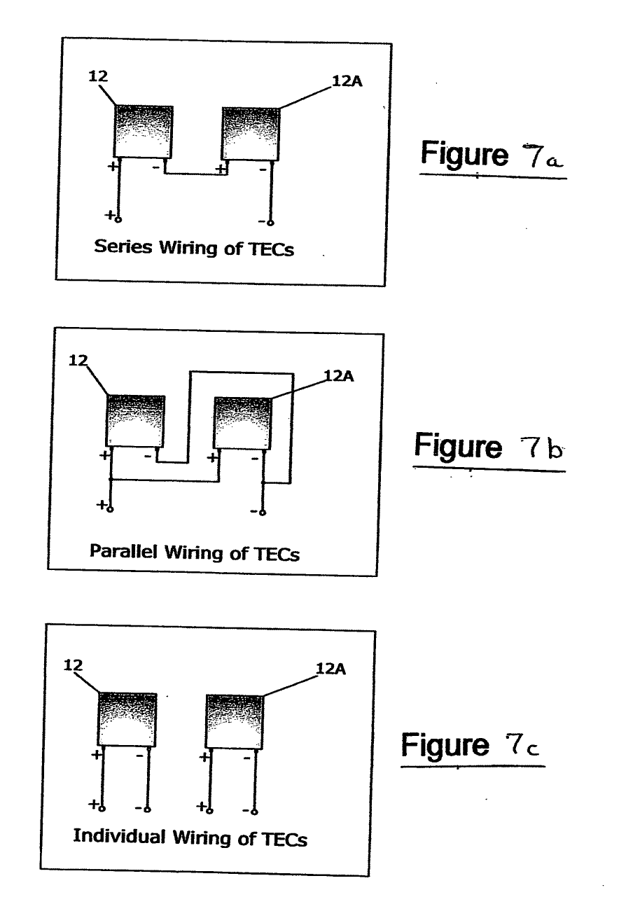

In a first embodiment of TEC connections as illustrated in FIG. 7a, a first TEC 12 is connected in series with a second TEC 12a, with the series-connected TECs being interposed between the HRM 10 and the cup 13 of FIG. 1. With this arrangement, heating is effected by use of the first TEC 12, and, for cooling, both the first TEC 12 and the second TEC 12a are employed. In this manner, the higher δT available in the cooling phase compensates for the slower cooling rate naturally encountered in TECs, and assists in making the thermal cycling reaction occur as rapidly as possible. Alternate connection embodiments of the first TEC 12 and the second TEC 12a are shown in FIG. 7b, a parallel connection, and FIG. 7c, an individual connection.

In general, the above-identified embodiments are not to be construed as limiting the breadth of the present invention. Modifications, and other alternative constructions, will be apparent which are within the spirit and scope of the invention as defined in the appended claims.

What is claimed is:

1. A thermal cycling apparatus comprising at least one reaction vessel and a thermoelectric cooler (TEC) arranged to provide both heating and cooling of the at least one reaction vessel.

2. Apparatus for conducting biological, chemical and biochemical processes comprising at least one reaction vessel arranged to be directly heated by a thermoelectric cooler.

3. Apparatus as set forth in claim 1 and wherein the TEC is a Peltier cell.

4. Apparatus as set forth in claim 2 and wherein the TEC is a Peltier cell.

5. Apparatus as set forth in claim 1 and wherein the TEC comprises a plurality of TECs in a series array.

6. Apparatus as set forth in claim 1 and arranged such that in operation a first side of the TEC is associated with the at least one reaction vessel and a second side thereof is arranged in use to be maintained at an intermediate temperature, which is intermediate the highest temperature and the lowest temperature used in a thermal cycling operation, and arranged for current to be supplied to the TEC in one direction whereby the first side becomes hotter than the second side, and then the current is supplied in the other direction whereby the first side becomes cooler than the second side.

7. Apparatus as set forth in claim 6 and arranged to carry out PCR and wherein the intermediate temperature is slightly below an extension temperature in the PCR cycle.

8. Apparatus as set forth in claim 7 and wherein the second side of the TEC is contiguous with a heat exchange block.

9. Apparatus as set forth in claim 8 and wherein the heat exchange block comprises a block of thermally conductive material having therein a channel adapted for the flow of a heat transfer liquid.

10. Apparatus as set forth in claim 8 and having a heat sink in communication with the heat exchange block.

11. Apparatus as set forth in claim 9 and wherein the channel is in serpentine form.

12. Apparatus as set forth in claim 9 and wherein the heat transfer liquid is deionized water with an antioxidant additive.

13. Apparatus as set forth in claim 6 and having a thermally conductive cup arranged to hold the at least one reaction vessel and wherein the first side of the TEC is contiguous with the cup.

14. Apparatus as set forth in claim 13 and wherein the first side of the TEC is contiguous with a base of the cup.

15. Apparatus as set forth in claim 10 and wherein the first side of the TEC is attached to a base of the cup.

16. Apparatus as set forth in claim 6 and having a temperature measuring device.

17. Apparatus as set forth in claim 16 and wherein the temperature measuring device is arranged for the control of electrical current to the TEC.

18. Apparatus as set forth in claim 6 and having an electrical control circuit in which there is means for detecting the absence and failure of a vessel and switching off current supply thereto.

19. Apparatus as set forth in claim 1 and comprising an array of reaction vessels and wherein there is one TEC per reaction vessel.

20. Apparatus as set forth in claim 19 and wherein the array is an 8×12 array or integer multiple thereof.

21. Apparatus as set forth in claim 1 and wherein the reaction vessel is a microtitre vessel.

22. Apparatus as set forth in claim 1 and wherein the reaction vessel has a reaction chamber comprising a tube of substantially capillary proportions.

23. Apparatus as set forth in claim 1 and wherein the reaction vessel is formed of a polymer loaded with a thermally conductive material.

24. Apparatus as set forth in claim 1 and wherein the reaction vessel has a lid, the lid having a translucent portion through which the reaction vessel contents can be monitored and which lid is arranged to be substantially contiguous with the reaction vessel contents in operation.

25. Apparatus as set forth in claim 1 and having an optical monitoring system arranged for monitoring the progress of a reaction within the reaction vessel.

26. Apparatus as set forth in claim 25 and wherein the optical monitoring system comprises a laser source, means for directing a laser into the reaction vessel, and a multi-anode photomultiplier tube for detecting resultant emitted light.

27. Apparatus as set forth in claim 25 and wherein the optical monitoring system comprises a printed circuit board (PCB) arranged for presentation above the reaction vessels, the PCB holding an array of light emitting diodes (LEDs) selected so as to be within the excitation spectrum of the reaction vessel contents under interrogation and arranged for the direction of light into the reaction vessel, the PCB also having a foramen arranged to permit the passage of vessel content light emission spectra, the system also comprising detector apparatus arranged to detect the emission spectra and filter means to block the path of excitation spectra to the detector.

28. Apparatus as set forth in claim 27 and wherein the LEDs are arranged to emit light at a wavelength of 470 nm or above.

29. Apparatus as set forth in claim 27 and comprising a Fresnel lens arranged to direct the light onto an XY scanning mirror set and thereby into a detector such as a photomultiplier tube, an avalanche photo-diode, charge couple device, light dependent resistor or a photovoltaic cell.

30. Apparatus as set forth in claim 27 and wherein the filter means comprises an optical filter placed across the foramen.

31. Apparatus as set forth in claim 27 and wherein the filter means comprises software associated with the detector.

32. A thermal cycling process performed in at least one reaction vessel and wherein a thermoelectric cooler (TEC) provides both heating and cooling of the said at least one reaction vessel.

33. A process comprising the steps of conducting any one of biological, chemical and biochemical processes in at least one reaction vessel, and directly heating the vessel by a thermoelectric cooler (TEC).

34. The process as set forth in claim 32 and wherein the TEC is a Peltier cell.

35. The process as set forth in claim 33 and wherein the TEC is a Peltier cell.

36. The process as set forth in claim 32 and wherein a first side of the TEC is associated with the at least one reaction vessel and a second side of the TEC is arranged in use to be maintained at an intermediate, which is a temperature intermediate the highest temperature and the lowest temperature used in the thermal cycling operation, a current being supplied to the TEC in one direction whereby the first side of the TEC becomes hotter than the second side, the current then being supplied to the TEC in the other direction whereby the first side of the TEC becomes cooler than the second side.

37. The process as set forth in claim 36 which is a PCR (polymerase chain reaction) process and wherein the intermediate temperature is slightly below the extension temperature in the PCR cycle.

38. The process as set forth in claim 32 and comprising the step of optically monitoring the progress of the PCR process.

39. The process as set forth in claim 36, wherein the TEC is a first TEC and comprising locating the first side of the first TEC in communication with a cup, positioning the at least one reaction vessel in the cup, locating the second side of the first TEC in a position contiguous with a second TEC such that the cup is contiguous with a cold side of the first TEC and a hot side of the first TEC is contiguous with a cold side of the second TEC.

40. The process as set forth in claim 39 comprising locating a hot side of the second TEC in a position contiguous with a third TEC.

41. The process as set forth in claim 39 comprising locating a hot side of the second TEC in a position contiguous with a heat exchange block.

42. The process as set forth in claim 41, comprising attaching the second side of the first TEC to the second TEC such that the cup is attached to the cold side of the first TEC and the hot side of the first TEC is attached to the cold side of the second TEC.

43. The process as set forth in claim 42 comprising attaching a hot side of the second TEC to a third TEC.

44. The process as set forth in claim 42 comprising attaching a hot side of the second TEC to a heat exchange block.
